(19) United States
(12) Patent Application Publication
(10) Pub. No.: US 2008/0109055 A1
(43) Pub. Date: May 8, 2008

(54) Implant for aortic dissection and methods of use

(75) Inventors: Edwin J. Hlavka, Minneapolis, MN (US); John Logan, Plymouth, MN (US)

Correspondence Address: O''Melveny & Myers LLP;IP&T Calendar Department LA-1118, 400 South Hope Street, Los Angeles, CA, 90071-2899, US

(73) Assignee: SAGE MEDICAL TECHNOLOGIES, INC.

(21) Appl. No.: 11/591,934

(22) Filed: Nov. 2, 2006

(51) Int. Cl. A61F 2/06

(52) U.S. Cl. 623/1.1

(57) ABSTRACT

Methods and devices for treating an aortic dissection having an entry point downstream of the takeoff of the left subclavian artery. The devices include a catheter that carries an endoluminal implant at a distal region of the catheter. The implant is a self-expanding tubular mesh or strutted stent. A capture sheath holds the stent in a compressed state for percutaneous delivery. The catheter is advanced to position the stent adjacent the entry point of the dissection. The stent is released by withdrawing the capture sheath. The stent expands to engage the intimal lining and press the intima into contact with the outer layers of the aorta and thereby promote healing of the dissection.

### FIELD OF THE INVENTION

The present invention relates generally to treatment of aortic dissections and treatment of type-B aortic dissections using a tubular implant to stabilize and remodel the tissues of the aorta.

### BACKGROUND

Aortic dissection most commonly occurs in patients between the age of 40 to 60 years old and is two or three times more frequent in men than women within this age group. Hypertension, a coexisting condition in 70% of the patients, is almost invariably the most important factor causing or initiating aortic dissection. Other risk factors that predispose a patient to develop aortic dissection include aortic dilation, aortic aneurysm, congenital valve abnormality, coarctation of aorta, and Marfan syndrome. These patients often present with sudden, severe, and tearing pain that may be localized in the front or back of the chest. Other symptoms include syncope, dyspnea, and weakness. These presentations are the consequence of intimal tear in the aorta, dissecting hematoma, occlusion of involved arteries, and compression of adjacent tissues. For example, patients may have neurological symptoms, such as hemiplegia, due to carotid artery obstruction, or paraplegia, due to spinal cord ischemia. Patients may also present with bowel ischemia or cardiac ischemia due to occlusion of major arteries by the dissecting aorta.

Aortic dissection can be classified by the Stanford method into type A and type B depending on the location and the extent of the dissection. Type A dissection, or proximal dissection, involves the ascending aorta. Type B dissection, or distal dissection, usually begins just downstream of the left subclavian artery, extending downward into the descending and abdominal aorta. If left untreated, the risk of death from aortic dissection can reach 35% within 15 minutes after onset of symptoms and 75% by one week.

Once diagnosed, aortic dissection is treated with immediate medical management aimed at reducing cardiac contractility and systemic arterial pressure, thereby reducing shear stress on the aorta. Beta-adrenergic blockers, unless contraindicated, are usually used to treat acute dissection. Surgical correction, including reconstruction of the aortic wall, is usually the preferred treatment for ascending aortic dissection (type A). Medical therapy is the preferred treatment for stable and uncomplicated distal aortic dissection (type B), unless there is clinical evidence of propagation, obstruction of major arterial branches, or impending aortic rupture in which case surgical correction is preferred. In-hospital mortality for medically treated patients with type B dissection is between 15 to 20 percent. Morbidity and mortality for surgical correction is not significantly better than medically treated patients. Currently, there is no good treatment for type B aortic dissection. A need for devices and methods therefore exists to treat patients suffering from Type B dissection.

### SUMMARY OF THE INVENTION

The present invention relates to devices and methods for treating aortic dissection and in particular type-B aortic dissection. Type-B dissections typically have an entry point immediately downstream of the takeoff of the left subclavian artery from the aorta. The device used herein is a catheter having a proximal end, a distal region, and a distal end. The catheter carries an endoluminal implant, commonly referred to as a stent, which comprises a porous mesh that is releaseably mounted on the distal region of the catheter. The implant is a generally cylindrical member having a length and the implant is expandable between a low-profile compressed state and an enlarged state. The implant may be pre-curved in the enlarged state. In the enlarged state, the implant has a proximal opening (downstream opening), a distal opening (upstream opening), and a lumen therebetween. The implant may also be equipped with a porous mesh or a textile covering a portion of the upstream region, the downstream region, or both the upstream and downstream regions of the implant. Further, the implant may be over-curved relative to the aorta in the region proximate to or upstream of the entry point of the dissection. Over-curvature ensures that the distal region of the implant adjacent the lesser curvature of the aorta achieves uniform wall contact with the lesser curvature of the aorta.

The methods of the present invention make use of a catheter with endoluminal implant or stent as described above. The catheter is generally introduced into the patient's aorta through an access site in the femoral artery. The catheter is advanced into the abdominal and thoracic aorta taking care not to enter the false lumen formed by the dissection. The catheter is advanced through the native lumen and positioned adjacent the entry point on the aorta. The self-expanding endoluminal implant is held in a collapsed state by an elongate capture sheath that extends proximal from the region that carries the implant. Once in place, the endoluminal implant is released by withdrawing the capture sheath. The implant assumes its enlarged, optionally pre-curved state and engages the endoluminal surface of the aorta.

The implant or stent is composed of a woven metal structure or strutted configuration, e.g., as produced by laser etching of a metal tube (e.g., stainless steel or nitinol) or weaving/braiding of a metal wire. In cases where the stent is pre-curved, the stent conforms substantially to the curvature of the aorta without distorting native anatomy. In cases where the stent is over-curved relative to the aorta in the region proximate to the entry point of the dissection, the upstream edge of the stent achieves uniform wall contact and does not lift away from the endoluminal surface of the lesser curvature. The upstream edge may also include an extension to assist in maintaining contact at the endoluminal surface of the lesser curvature. The woven or strutted configuration is sufficiently porous to allow perfusion of arteries that branch from the aorta, e.g., the intercostal arteries, celiac trunk, superior mesenteric artery, renal arteries, left subclavian artery, left common carotid, and inferior mesenteric artery.

In cases where the stent is covered at its upstream, downstream, or upstream and downstream ends with a textile, a porous textile is used. The textile is selected from various biocompatible textiles that promote tissue in-growth to promote healing. The textile extends only over limited parts of the stent, e.g., over the portion of the stent that engages the entry point of the dissection and/or re-entry point of the dissection. The remainder of the stent is free of covering to allow perfusion of arteries that branch from the aorta.

### BRIEF DESCRIPTION OF THE DRAWINGS

FIG. 1A depicts the anatomy of the aorta.

FIGS. 1B and 1C depict the various tissue layers of the aorta in cross-section.

FIG. 1D depicts the aorta in cross-section with dissection.

FIG. 2 depicts an aorta with the beginning stages of a dissection.

FIG. 3 depicts the aorta of FIG. 2 having a dissection that has progressed downstream along a length of the aorta.

FIG. 4A depicts the aorta of FIG. 3 having a dissection that has progressed to a re-entry point downstream in the aorta.

FIG. 4B is a cross-section view of the aorta in FIG. 4A through section line A-A.

FIG. 5 depicts a catheter for use in repair of aortic dissection as described herein.

FIG. 6 depicts the catheter of FIG. 5 with the implant partially deployed.

FIG. 7 depicts the catheter of FIG. 5 advanced into the thoracic aorta.

FIG. 8 depicts the deployment of the implant to close the entry point of the aortic dissection.

FIG. 9 depicts the implant of FIG. 8 deployed to close an aortic dissection.

FIG. 10 depicts a pre-curved or over-curved implant for use herein to treat aortic dissection.

FIG. 11 depicts an implant having portions of textile covering for use herein to treat aortic dissection.

### DETAILED DESCRIPTION

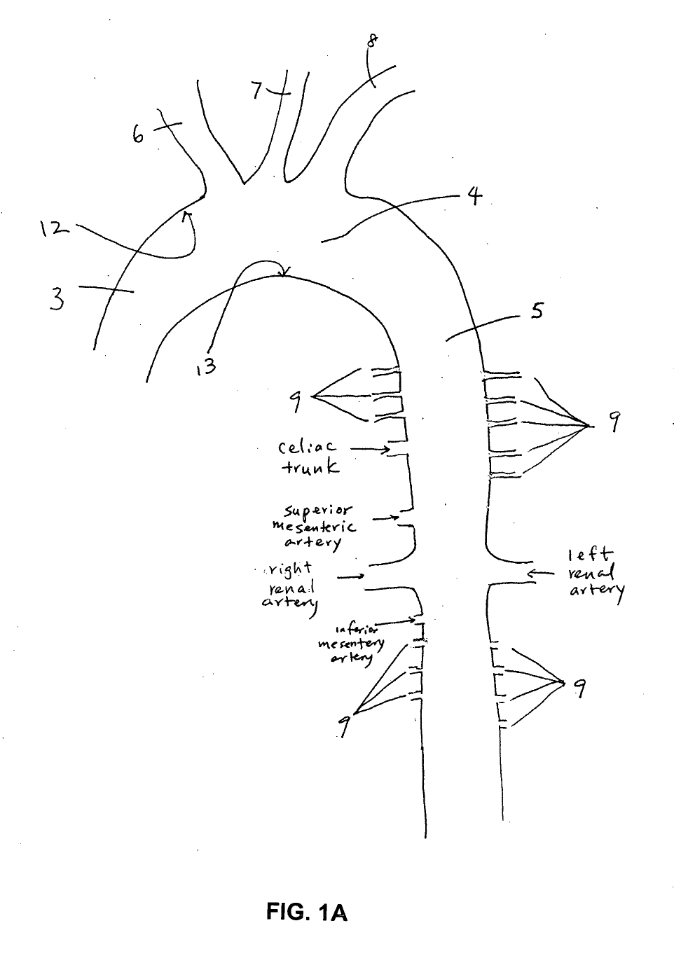

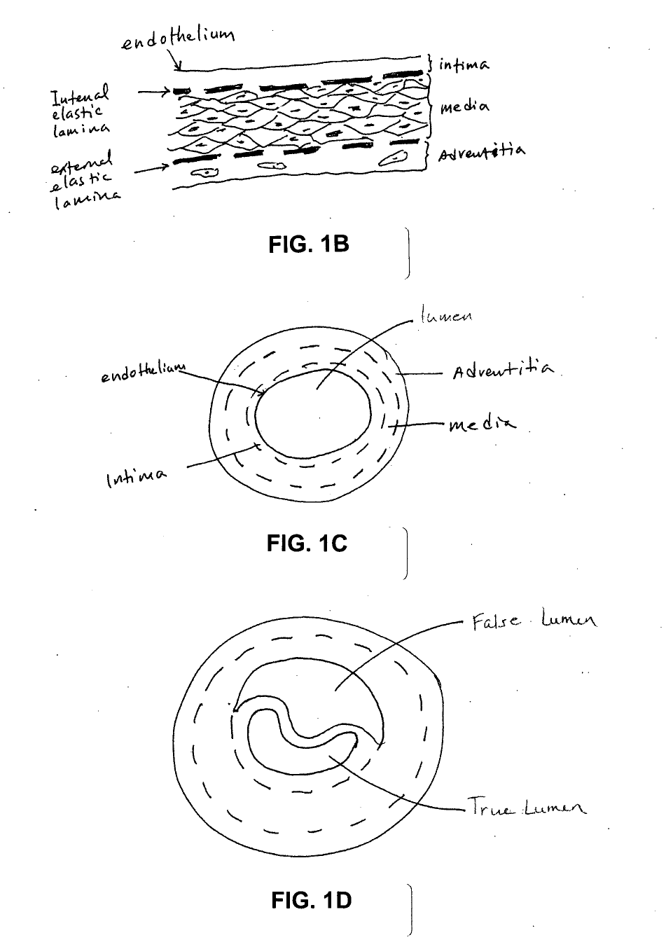

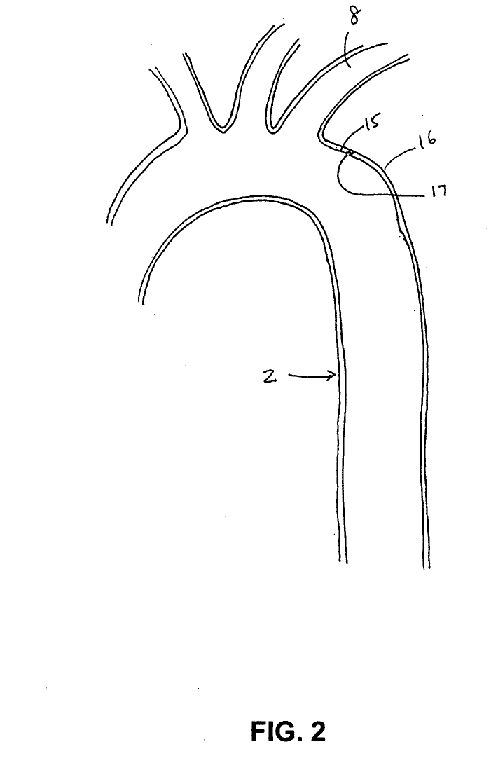

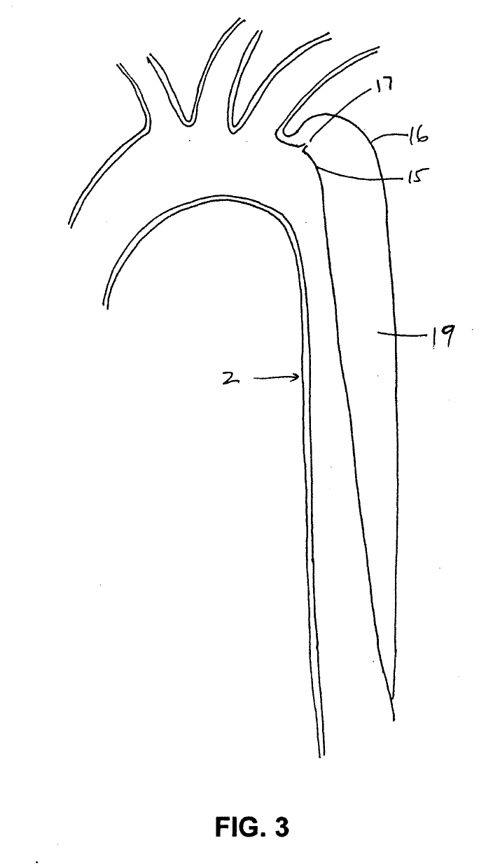

The aorta of a normal individual is depicted in FIG. 1A. Aorta 2 is anatomically designated as having ascending aorta 3, aortic arch 4, and descending aorta 5. Aortic arch 4 includes greater curvature 12 and lesser curvature 13. A number of arteries branch from aorta 2 and supply blood to many of the body's vital organs. For example, innominate artery 6, left common carotid artery 7, and left subclavian artery 8 supply blood to various regions of the brain. If blood flow to any of these arteries is interrupted, stroke may result. Intercostal arteries 9 branch from descending aorta 5 and supply blood to various regions of the spine and spinal cord. Interruption of blood flow in the intercostal arteries can result in paraplegia. The superior and inferior mesenteric arteries supply blood to the intestines, the celiac artery supplies blood to the liver, and the renal arteries supply blood to the kidneys. Interruption of blood flow in any of these arteries can have devastating results.

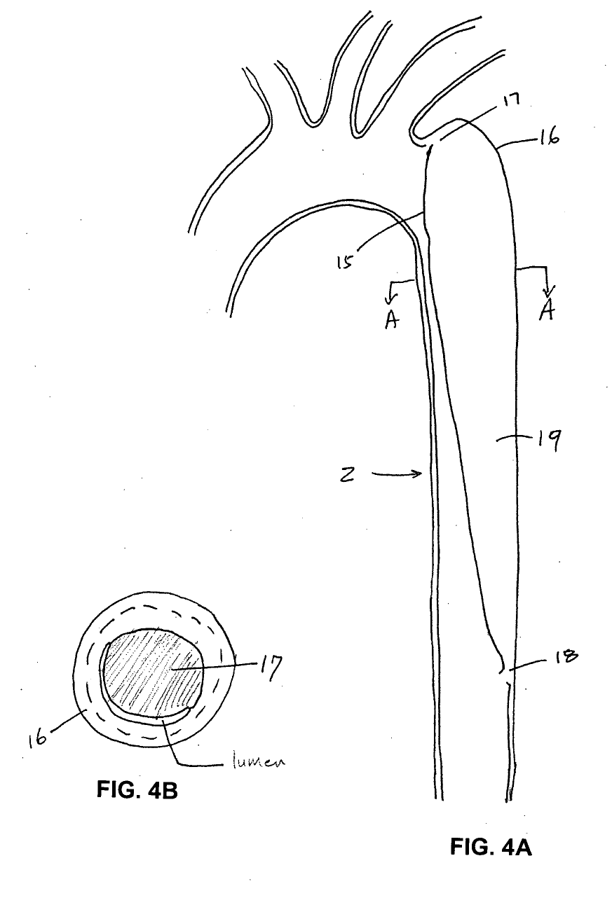

FIG. 2 illustrates the initiation of an aortic dissection. The most common aortic dissections occur near the ostium of left subclavian artery 8, just downstream where blood passing along the greater curvature of the arch impacts the intimal lining of the aorta at the takeoff of the left subclavian artery. Intimal lining 15 begins to tear away from outer layers 16 of the aorta, which layers include the media and adventitia (see FIGS. 1B, 1C, and 1D). Entry point 17 opens as a result of tearing, which creates a chamber between torn intima 15 and outer layers 16. The chamber receives and traps blood, and as the pressure builds within the chamber, blood flow causes the tear to progress downstream as depicted in FIG. 3. As intima 15 pulls away from outer layers 16, a false lumen 19 is formed that progresses downstream in descending aorta 5 as depicted in FIG. 4A. Re-entry point 18 forms where the intima tears from itself to allow blood to re-enter the natural lumen.

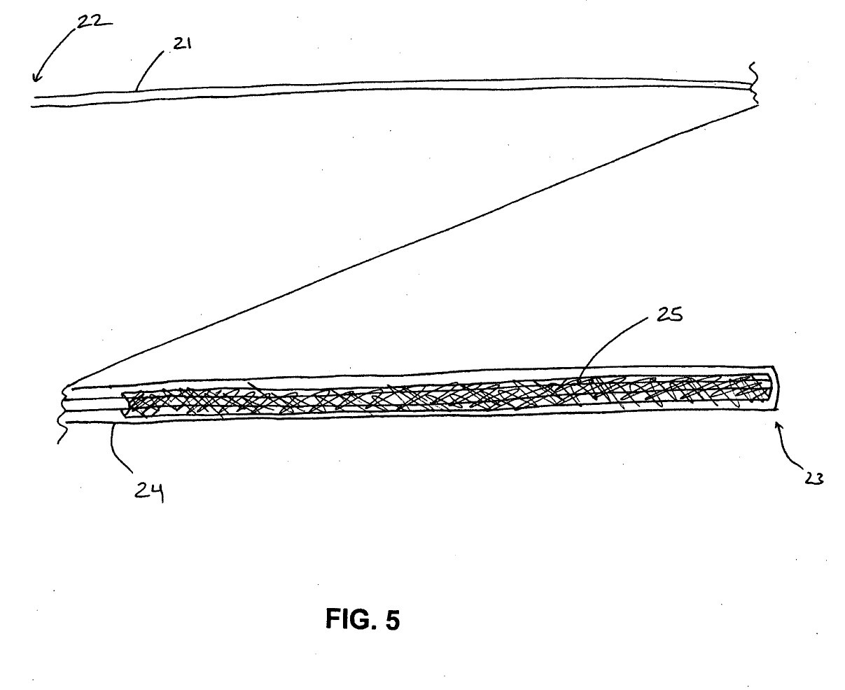

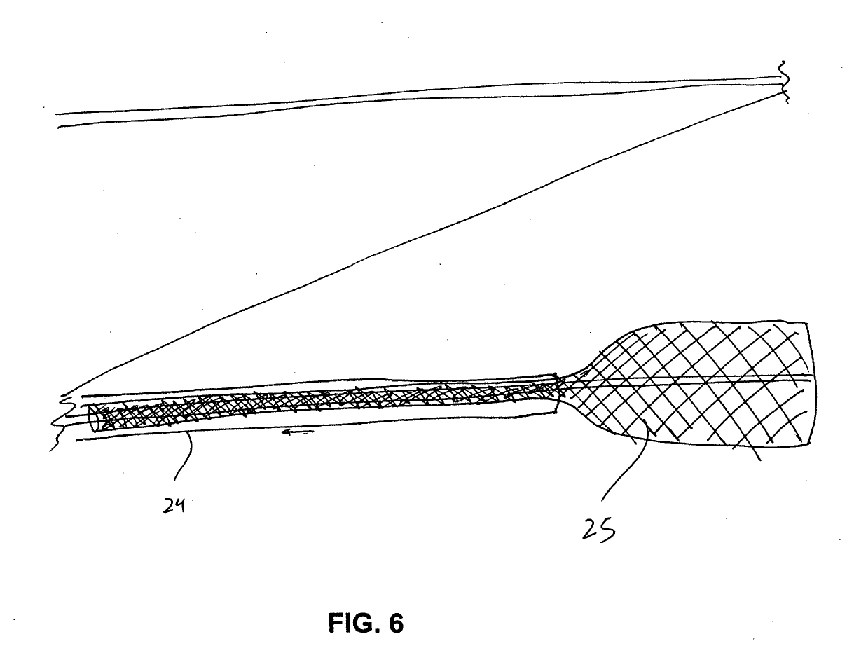

A catheter for aortic dissection repair is depicted in FIG. 5. Catheter 21 is an elongate tubular member and has proximal end 22, distal end 23, and is adapted and sized for advancement into the aorta through a femoral artery access site. Catheter 21 may include a lumen that extends proximally from one or more ports at a distal region for administering pharmaceutical agents. A self-expanding stent 25 is loaded on the distal region or distal end of catheter 21. Stent 25 is held in a low profile configuration by sheath 24, which is an elongate tubular member operable from the proximal end of catheter 21. Sheath 24 is withdrawn proximally to uncover and thereby release stent 25 as illustrated in FIG. 6. As stent 25 is released, it expands to an enlarged state adapted to engage the endoluminal surface of the aorta.

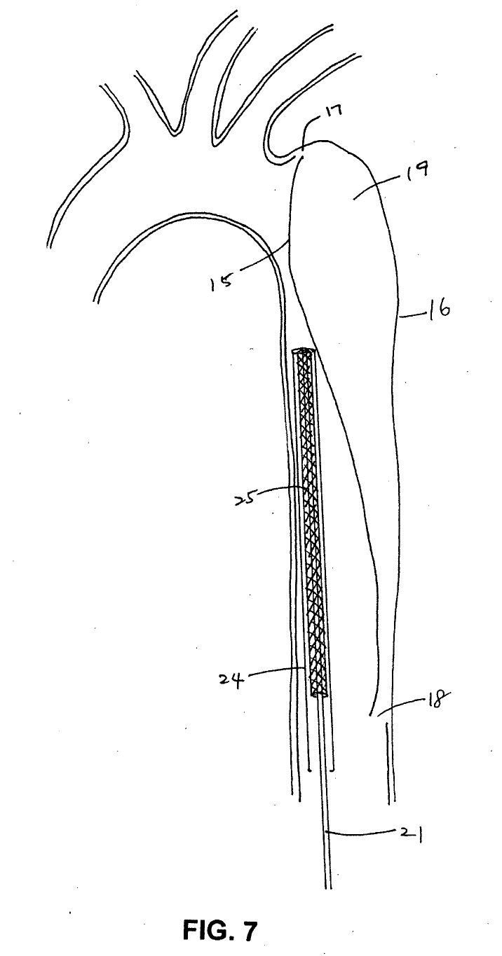

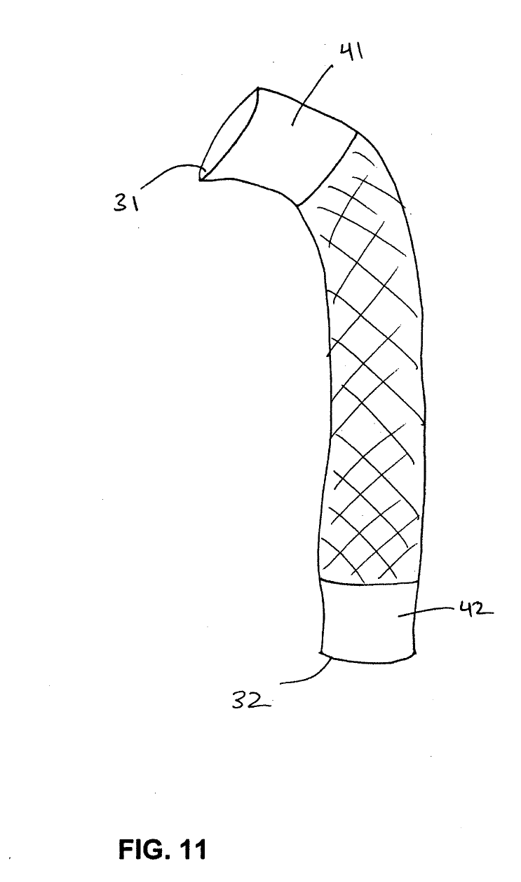

In use, stent delivery catheter 21 is advanced through a femoral access site into the descending aorta as illustrated in FIG. 7. Catheter 21 is advanced retrograde past the downstream edge of torn intima 15 so that catheter 21 remains within the native lumen (not within the false lumen). Distal end 23 of catheter 21 is positioned adjacent entry point 17 of the aortic dissection. The procedure may be conducted using standard fluoroscopic visualization techniques to align catheter 21 with anatomical landmarks visible by angiography. One or more fluoroscopic markers may be included on catheter 21, on the distal region or distal end 23 of catheter 21, on covering 41 (see FIG. 11), on covering 42 (see FIG. 11), and/or on stent 25 for purposes of alignment. The takeoff of left subclavian artery 8 or entry point 17 are among anatomical landmarks useful for alignment.

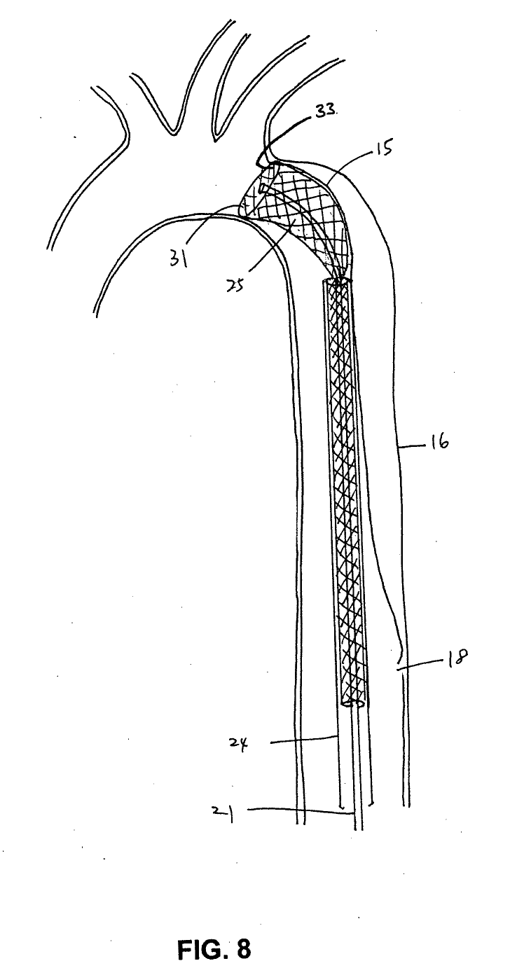

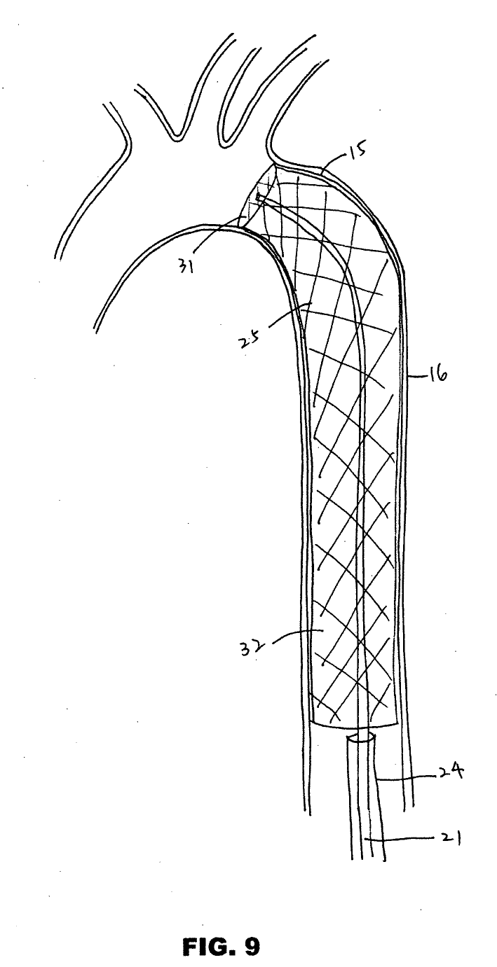

After distal end 23 of catheter 21 is aligned with entry point 17 at the most upstream edge of the intimal tear, sheath 24 is withdrawn proximally to release stent 25 as shown in FIG. 8. Stent 25 expands to engage intima 15 and then displace intima 15 until it makes contact again with outer layers 16 of aorta 2. Intima 15 is thereby pressed into contact with the outer layers of the aorta. Stent 25 contacts the intimal tear to close entry point 17 at a first position 33 on the circumference of the upstream region (distal region) or upstream end of stent 25. A second position 31 on the circumference of the upstream region (distal region) or upstream end of stent 25, approximately 180° relative to first position 33, engages the endoluminal surface of the aorta at the lesser curvature. As will be explained in greater detail below, stent 25 may be pre-curved, and in certain cases over-curved relative to the curvature of the aorta so that second position 31 on stent 25 achieves uniform wall contact along the endoluminal surface at the lesser curvature.

As stent 25 displaces intima 15 toward outer layers 16 of the aorta, blood is purged from the false lumen and the false lumen is gradually closed. This process continues as shown in FIG. 8 as sheath 24 is withdrawn proximally until proximal end 32 (the downstream end) of stent 25 is released in the downstream region of the descending aorta as shown in FIG. 9. Proximal end 32 of stent 25 expands to close re-entry point 18. Substantially all blood is forced out of the false lumen created by the aortic dissection. Catheter 21 and sheath 24 may then be withdrawn from the aorta and removed from the patient. With time, any remaining blood trapped between layers of the vessel will be removed by the healing process as the aorta is remodeled by re-attachment of intimal layer 15 to outer layers 16. The woven or strut pattern of stent 25 moreover is sufficiently porous to allow perfusion of intercostal arteries 9 and other arteries that branch from the aorta in the region now covered by the stent.

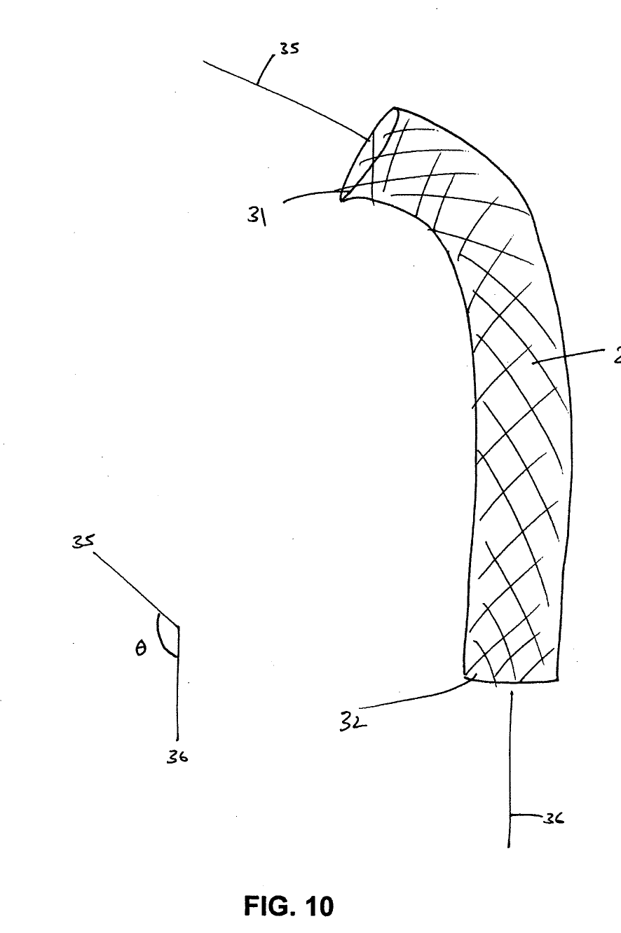

The subject matter herein may be implemented so that stent 25 achieves uniform wall contact, especially where the stent contacts the lesser curvature of the aorta arch, and conforms to the curvature of the aorta without distorting native anatomy. These objectives may be accomplished using a pre-curved stent as depicted in FIG. 10. The upstream or distal end 31 of stent 25 has longitudinal axis 35. The downstream or proximal end 32 of stent 25 has longitudinal axis 36. Axis 35 and axis 36 meet at angle theta. As described herein, it is understood that stent 25 may desirably be implemented with pre-curved angle theta of 145° or less, 140° or less, 130° or less, 120° or less, 110° or less, 100° or less, 90° or less, 80° or less, 70° or less, 60° or less, or 50° or less. By using a stent that is over-curved relative to the aorta in the region proximate to the entry point of the dissection 17 (see FIGS. 7, 8 and 9), the leading edge 31 of stent 25 achieves uniform wall contact with the endoluminal surface of the lesser curvature of the aorta. Without uniform wall contact at leading edge 31, blood flow along the lesser curvature will impact leading edge 31, pulling the leading edge away from the lesser curvature and causing blood flow turbulence.

The devices may also include a portion of a textile material on the distal region (upstream region), the proximal region (downstream region), or both the proximal and distal regions. A stent having textile 41 and 42 on distal and proximal regions is illustrated in FIG. 11. Textile 41 at the upstream end of stent 25 may be disposed on the outer circumference of metal stent 25. Alternatively, textile 41 at the upstream end of stent 25 may be disposed on the inner circumference of metal stent 25. Textile 41 may extend downstream for a length of 1 cm, 2 cm, 3 cm, 4 cm or more. Textile 41 may be composed of Dacron, nylon, Teflon (PTFE), expanded PTFE (ePTFE), urethanes (Lycra Spandex), polypropylene, silicone, biodegradable synthetics, such as polyglycolide (PGA), polylactide (PLA), biologics, and composites, or any other biocompatible material suitable for intravascular use. Coatings may be added to affect physiologic response, e.g., blood clotting and healing. For instance, prothrombin, which induces clotting, may be coated on the textile positioned near or adjacent the entry tear. Coatings may be added to resist thrombogenesis, e.g., heparin coating. For instance, heparin might be used on the un-covered portion of the stent that is distal to the entry tear to prevent clotting around the intercostals. Textile 41 is advantageously composed of a porous mesh material having a pore size of greater than 50 microns, greater than 60 microns, greater than 70 microns, greater than 80 microns, greater than 90 microns, greater than 100 microns, greater than 110 microns, or greater than 120 microns. At the same time, pore size will advantageously be less than 2000 microns, less than 1500 microns, less than 1000 microns, less than 750 microns, less than 500 microns, or less than 250 microns. The porosity of the textile may also be described with reference to flow rate. Porosity will be chosen to allow a flow rate of greater than 800 mL/cm2·min at 120 mmHg, greater than 850 mL/cm2·min at 120 mmHg, greater than 900 mL/cm2·min at 120 mmHg, or greater than 1000 mL/cm2·min at 120 mmHg. Porosity will be chosen to allow a flow rate of less than 20,000 mL/cm2·min at 120 mmHg, less than 18,000 mL/cm2·min at 120 mmHg, less than 15,000 mL/cm2·min at 120 mmHg, or less than 10,000 mL/cm2·min at 120 mmHg. The textile may have the ability to promote in-growth of vascular cells to remodel the intimal lining for long-term healing.

Textile 42, when present, at the downstream end of stent 25 may be disposed on the outer circumference of metal stent 25. Alternatively, textile 42 at the downstream end of stent 25 may be disposed on the inner circumference of metal stent 25. Textile 42 may extend upstream for a length of 1 cm, 2 cm, 3 cm, 4 cm, or more. Textile 42 may be composed of Dacron, nylon, Teflon (PTFE), expanded PTFE (ePTFE), urethanes (Lycra Spandex), polypropylene, silicone, biodegradable synthetics, such as polyglycolide (PGA), polylactide (PLA), biologics, and composites, or any other biocompatible material suitable for intravascular use. Coatings may be added to affect physiologic response, e.g., blood clotting and healing. For instance, prothrombin, which induces clotting, may be coated on the textile positioned near or adjacent the entry tear. Coatings may be added to resist thrombogenesis, e.g., heparin coating. For instance, heparin might be used on the un-covered portion of the stent that is distal to the entry tear to prevent clotting around the intercostals. Textile 42 is likewise advantageously composed of a porous mesh material having pore sizes and flow characteristics in the ranges listed above for textile 41. Textile 42 may also have the ability to promote in-growth of vascular cells to remodel the intimal lining for long-term healing at the reentry point.

The working length of catheter 21 will generally be between 30 and 100 centimeters, preferably approximately between 50 and 80 centimeters. The outer diameter of the catheter 21 shaft will generally be between 1 French and 8 French, preferably approximately between 1.5 French and 4 French. The outer diameter of sheath 24 will generally be between 10 and 22 French, preferably approximately between 12 and 16 French. Stent 25 may vary in length but is generally approximately 5 cm to 30 cm, preferably approximately 10 cm to 20 cm. The foregoing ranges are set forth solely for the purpose of illustrating typical device dimensions. The actual dimensions of a device constructed according to the principles of the present invention may obviously vary outside of the listed ranges without departing from those basic principles.

Although the foregoing invention has, for the purposes of clarity and understanding, been described in some detail by way of illustration and example, it will be obvious that certain changes and modifications may be practiced that will still fall within the scope of the appended claims. For example, the devices and features depicted in any figure or embodiment can be used in any of the other depicted embodiments.

What is claimed is:

1. A method for treating an aortic dissection having an entry point downstream of the takeoff of the left subclavian artery, the method comprising the steps of:
providing a catheter having a proximal end, a distal region, and a distal end, the catheter having an endoluminal implant releasably mounted in the distal region, the endoluminal implant comprising a generally cylindrical member having a length and being expandable between a compressed state and a pre-curved enlarged state, the cylindrical member having a proximal opening, a distal opening, and a lumen therebetween;
advancing the distal region of the catheter to a position near the entry point on the aorta; and
releasing the endoluminal implant to assume its pre-curved enlarged state and engage at least a portion of the endoluminal surface of the aorta,
wherein a region of the endoluminal implant that defines the distal opening is over-curved relative to the aorta in the region proximate to the entry point of the dissection so that a portion of the region of the endoluminal implant adjacent the lesser curvature of the aorta achieves uniform wall contact with the lesser curvature of the aorta.

2. The method of claim 1, wherein the generally cylindrical member has a turning angle of greater than 90 degrees.

3. The method of claim 1, wherein the step of releasing the endoluminal implant is performed so that the endoluminal implant assumes a pre-curved enlarged state that conforms to the curvature of the aorta.

4. The method of claim 1, wherein the step of releasing the endoluminal implant is performed so that the endoluminal implant achieves uniform wall contact along the length of the endoluminal implant without distorting native anatomy.

5. The method of claim 1, wherein the step of releasing the endoluminal implant is performed so that the endoluminal implant engages the endoluminal surface of the aorta.

6. The method of claim 1, wherein the distal end of the endoluminal implant prevents blood flow through the entry point.

7. The method of claim 1, wherein the endoluminal implant allows perfusion of arteries that branch from the aorta.

8. The method of claim 1, wherein the endoluminal implant further comprises a distal covering of a porous textile bonded to the endoluminal implant.

9. The method of claim 8, wherein the porous textile has a pore size that allows a flow rate of greater than 800 mL/cm2·min at 120 mmHg.

10. The method of claim 1, wherein the endoluminal implant comprises a structure selected from the group consisting of a porous mesh, a braided wire, and a laser etched metal tube.

11. A method for treating an aortic dissection having an entry point downstream of the takeoff of the left subclavian artery, the method comprising the steps of:
providing a catheter having a proximal end, a distal region, and a distal end, the catheter having an endoluminal implant releasably mounted in the distal region, the endoluminal implant comprising a generally cylindrical member having a length and being expandable between a compressed state and an enlarged state, the cylindrical member having a proximal opening, a distal opening, and a lumen therebetween, the endoluminal implant further comprising a distal covering of a porous textile bonded to the cylindrical member, the porous textile having a pore size that allows a flow rate of greater than 800 and less than 20,000 mL/cm2·min at 120 mmHg;
advancing the distal region of the catheter to a position adjacent the entry point on the aorta; and
releasing the endoluminal implant to assume its enlarged state so that the distal covering engages the entry point,
wherein the distal end and distal covering of the tubular endoluminal implant prevents blood flow through the entry point and the cylindrical member allows perfusion of arteries that branch from the aorta.

12. The method of claim 11, wherein the generally cylindrical member has a turning angle of greater than 90 degrees.

13. The method of claim 11, wherein the step of releasing the endoluminal implant is performed so that the endoluminal implant assumes a pre-curved enlarged state that conforms to the curvature of the aorta.

14. The method of claim 11, wherein the step of releasing the endoluminal implant is performed so that the endoluminal implant achieves uniform wall contact along the length of the endoluminal implant without distorting native anatomy.

15. The method of claim 11, wherein the step of releasing the endoluminal implant is performed so that the endoluminal implant engages the endoluminal surface of the aorta.

16. The method of claim 11, wherein a region of the endoluminal implant that defines the distal opening is over-curved relative to the aorta in the region proximate to the entry point to the dissection so that a portion of the region of the endoluminal implant adjacent the lesser curvature of the aorta achieves uniform wall contact with the lesser curvature of the aorta.

17. The method of claim 11, wherein the distal opening is defined by the circumference of the cylindrical member, the cylindrical member further comprising an extension along a circumferential region.

18. The method of claim 11, wherein the endoluminal implant comprises a structure selected from the group consisting of a porous mesh, a braided wire, and a laser etched metal tube.

19. The method of claim 11, wherein the porous textile has a pore size that allows a flow rate of greater than 900 and less than 18,000 mL/cm2·min at 120 mmHg.

20. The method of claim 11, wherein the porous textile has a pore size that allows a flow rate of greater than 1000 and less than 15,000 mL/cm2·min at 120 mmHg.
